(19) United States
(12) Patent Application Publication
(10) Pub. No.: US 2017/0135596 A1
(43) Pub. Date: May 18, 2017

(54) Bioelectrical Signal Sensor

(71) Applicant: Suzhou Greentek Pty. Ltd., Suzhou City, Jiangsu Province (CN)

(72) Inventor: Ansheng Fan, Suzhou (CN)

(21) Appl. No.: 15/322,231

(22) PCT Filed: Aug. 26, 2014
(86) PCT No.: PCT/CN2014/085141

§ 371 Date: Dec. 27, 2016

(30) Foreign Application Priority Data
Jun. 28, 2014 (CN) 201410302153.1

(51) Int. Cl. A61B 5/0478

(52) U.S. Cl. CPC A61B 5/0478; A61B 2562/0209

(57) ABSTRACT

The present disclosure provides a bioelectrical signal sensor, which comprises electrode(s), electrolyte, a cavity for containing—electrolyte, and porous column(s). The cavity has a sealed end at one end and communicates at the other end with an electrolyte inlet end of each porous column, with the other end of each porous column being a working end in contact with an organism. At least a portion of the electrode is located in the electrolyte. The electrode is an electrical conductor, and the cavity is an electrical conductor or an electrical insulator. The bioelectrical signal sensor has a number of advantages, including: simple structure, reliable contact, accurate positioning, low and stable electrode-skin impedance, low noise and small artifacts, ease and comfort for use, ability for long-term recycling, and suitability for a variety of bioelectricity-related recording, measurement and stimulation applications, such as electroencephalogram (EEG) measurements.

### CROSS-REFERENCE TO RELATED PATENT APPLICATION(S)

The present disclosure is part of U.S. national stage of PCT patent application PCT/CN2014/085141, filed on 26 Aug. 2014, which claims the priority benefit of China Patent Application No. 201410302153.1, filed on 28 Jun. 2014, which is incorporated by reference in its entirety.

### TECHNICAL FIELD

The present disclosure, relating to a bioelectrical signal sensor, belongs to the field of bioelectrical technology, and is widely used in bioelectrical recording, measurement and stimulation, including high-density bioelectrical measurement, medical equipment, mobile devices, home health care, psychological cognition, games, brain-computer interface, and rehabilitation training, especially suitable for electroencephalogram (EEG) measurements.

### BACKGROUND

With the development of science and technology, bioelectricity has been widely used in monitoring and diagnosing a variety of neurological diseases, and rehabilitation equipment with bioelectrical feedback. Bioelectrical measurement is also an important experimental means of cognitive psychology researches. Since EEG signal is very weak, generally at the level of microvolt, accurate acquisition of EEG signals requires precise and reliable equipment systems. An EEG measurement equipment system generally includes EEG sensors, a signal amplifier, a signal processing system, and a signal display and recording system, in which EEG sensor is the key component to acquire weak EEG signals.

The transmission of EEG signals is carried out in the intercellular fluid (as electrolyte), essentially being the transmission of a weak ionic electrical signal in the electrolyte. With the poor electrical conductivity of the human skin or hair, there are too high impedance and instable contact between the EEG electrode and the human skin (hair), and the EEG signal will be greatly attenuated after passing the electrode-skin interface, directly affecting the acquisition of EEG signals. In order to accurately measure EEG without using an invasive electrode, conductive pastes or gels are often used to provide an electrolyte environment and form an ionic conductive path. The conductive pastes or gels help bioelectricity spread from inside of the human body to the measuring electrode, form a good electronic conductor/ionic conductor interface between the electrode and the skin, and convert the weak ionic electrical signals (in the human body) into electronic electrical signals for measurement. This extends the ionic channel to the electrode contact, and makes the electrode-skin impedance low and stable. As a result, satisfactory signal is recorded. This electrode technology of applying conductive pastes or gels between the electrode and the skin is the currently widely used, referred as wet electrode. Although the wet electrode can reduce the impedance between the electrode and the skin to obtain accurate EEG signals, it has a number of drawbacks described below.

1. The need for professional stuff to apply the conductive pastes or gels limits the family, games and other scenarios applications; besides, applying conductive pastes or gels often cost much time.

2. The conductive pastes or gels are prone drying or falling off after a long time, resulting in measurement interruption, therefore not suitable for long-term EEG recording for rehabilitation equipment.

3. The conductive pastes or gels spread over subjects' head, and need to wash off after the test, having an uncomfortable feeling; in addition, the operator needs to clean the electrode with conductive pastes or gels, which brings some trouble.

4. For the high-density EEG measurement (64 channels or more), injecting conductive gels costs a long time, and the adhesive is prone to being accidentally not injected to an individual electrode, needing a check and re-injection, which undoubtedly brings a lot of trouble to the high-density EEG measurement.

In order to overcome the above drawbacks of the wet electrode, new electrode technologies have being exploring in recent years, i.e., the electrode technologies needing no conductive paste or gels. These new electrode technologies are explored in two directions.

The first direction relates to using fiber fabrics, hydrogels and other water-retaining materials instead of the conductive pastes or gels. The advantage is that the subject is more accepted to it than the wet electrode, avoiding the inconvenience of applying the conductive pastes or gels. The disadvantage is that these fiber fabrics, hydrogels and other water-retaining materials, generally a flexible whole piece, are not easy to penetrate through the hair like the conductive pastes or gels, difficult to well contact the skin, and impossible to form a stable ionic conductor path. Therefore, sometimes there will be high or unstable electrode impedance, which affects the signal quality. In order to solve the above mentioned problem, rigid tubes are often used as supporting. To ensure penetrate through hair effectively, the rigid tubes must be small. Even worse, the tube walls occupy a certain volume of the small tubes. However, it will cause the ionic path to be too small to make the impedance instable in the practical applications. Besides, although the contact problem is basically solved by using the small tubes as supporting, the water-absorbing materials tends to shrink into the small tubes due to repeated swelling and cleaning, resulting in poor contact between the ionic conductor materials and the scalp, which affects the long-term use of the electrode. In addition, such a water-absorbing material, after the loss of the electrically conductive liquid in the cavity, is difficult to be replenished with the electrically conductive liquid and other electrolytes, and thus cannot be long-term recycled.

The second direction relates to developing the dry electrode technology, namely, the electronic conductor of dry electrode contact directly with the scalp without any electrolytes such as conductive pastes or gels, hydrogels and water-absorbing fibers. Due to elimination using electrolytes (e.g., conductive pastes or gels, hydrogels), it is very convenient for electrodes placement. The g.SAHARA dry electrodes (g.tek company) have become available on the market. The g.SAHARA dry electrodes comprise multiple-tips arranged in the end of the dry electrode, which can penetrate through the hair like combs and then contact directly with the scalp. However, ionic paths cannot be established between the electrode and the scalp due to no electrolyte, which makes the electrode-skin impedance very high. In order to overcome the shortcoming of high impedance, this product has a pre-amplifier packaged in the back of each electrode, which undoubtedly increases the complexity of production. Another drawback of the dry electrode is that the rigid multiple-tips easily leads to pain. WO2013/142316 A1 uses active small claw-shape dry electrode, so that the electrode gently penetrates through the hair and can exert a certain pressure to the scalp, while subjects do not feel pain. The prior art of these dry electrodes, all employing the solid electronic conductor in direct contact with the skin, fails to establish an ideal ionic path; this electrode-skin contact will result in high and unstable electrode impedance, bringing noise and unstable signals.

The present disclosure, aiming at overcoming the drawbacks of the prior art bioelectrical signal sensor technology, still uses the principle of extending the ionic path to the skin, but does not need to apply the conductive gels or pastes, as convenient as the dry electrode.

### SUMMARY

A purpose of the present disclosure is to provide a bioelectrical signal sensor, which has simple structure, reliable contact, accurate positioning, low and stable electrode-skin impedance, low noise and artifacts. The proposed bioelectrical signal sensor is easy and comfortable to use, can be long-term recycled, and is suitable for a variety of bioelectricity-related recording, measurement and stimulation applications.

The present disclosure adopts a number of technical solution as described below.

A bioelectrical signal sensor may include: electrode(s), electrolyte, and a cavity for containing electrolyte, as well as porous column(s); the cavity has a sealed end at one end and communicates at the other end with the electrolyte inlet end of the porous column(s), with the other end of the porous column being a working end in contact with an organism; at least a portion of the electrode is immersed in the electrolyte; the electrode is an electrical conductor, and the cavity is an electrical conductor or an electrical insulator.

In the bioelectrical signal sensor, the porous column(s) may be made of a porous ceramic material or a porous ceramic composite.

In the bioelectrical signal sensor, the electrical conductor may be a conductor made of an electrically conductive material, an electrically insulating material plated on the surface with an electrically conductive material, or a composite conductor of electrically conductive material(s) and electrically insulating material(s).

In the bioelectrical signal sensor, the electrode and the cavity may be integrally one-time formed of the electrically conductive material, or ay be integrally one-time formed of the electrically insulating material and plated on the surface with an electrically conductive coating.

In the bioelectrical signal sensor, the sealed end of the cavity may be a sealing cover, which may be hermetically and detachably fixed to the body of the cavity. The electrolyte inlet end of the porous column may be fixed to a mounting hole in the end face of the cavity.

In the bioelectrical signal sensor, the sealed end of the cavity may be a sealing cover, which may be integrally bonded to or welded to or one-time formed with the body of the cavity. The electrolyte inlet end of the porous column may be detachably fixed to the mounting hole in the end face of the cavity, preferably by a scarf joint or threaded connection.

In the bioelectrical signal sensor, the cavity may be divided into an upper portion and a lower portion that are connected by a detachable seal. The upper portion of the cavity may be integrally bonded to or welded to or one-time formed with the sealing cover. The lower portion of the cavity may be fixed to the electrolyte inlet end of the porous column.

In the bioelectrical signal sensor, the porous column may be a tapered column, whose working end in contact with an organism is smaller than the electrolyte inlet end.

In the bioelectrical signal sensor, the sealing cover may be provided with an electrolyte supply hole and a hole lid.

In the bioelectrical signal sensor, the electrode may be an independent electrical conductor connected with an electrode wire, an electrically conductive end of the electrode wire exposed from the inner wall of the cavity, or an electrically conductive inner wall of the cavity to which an electrical wire is connected. A part of the electrode or the electrode wire may be hermetically fixed on the cavity, and other part or all the other part of the electrode may be in contact with the electrolyte.

In the bioelectrical signal sensor, the electrode may be fixed to the sealed end of the cavity by injection molding, or may be bonded to the sealed end of the cavity.

In the bioelectrical signal sensor, the electrically conductive material may be selected from gold, silver, silver/silver chloride, electrically conductive silicone rubber, an electrically conductive polymer, an electrically conductive carbon material, and a composite material thereof.

In the bioelectrical signal sensor, the electrically insulating material may be selected from plastic, rubber, and a composite material thereof.

In the bioelectrical signal sensor, the electrode may be conductive liquid(s), or conductive gel(s), or a combination thereof.

The present disclosure provides a number of remarkable beneficial effects as described below.

1. The present disclosure provides a bioelectrical signal sensor, which uses the design of a tapered porous ceramic column. On one hand, the tapered porous ceramic column can quickly penetrate through the hair to contact directly with the scalp, thus overcoming the impact of hair, keeping the porous column in good contact with the scalp, providing an electrolyte path, reducing the electrode-scalp impedance, and improving the signal to noise ratio, not requiring an electrically conductive paste. Other technologies use flexible water-absorbing fabrics, water-absorbing fibers and other electrolyte materials as the ionic conductive path, which cannot penetrate well through the hair to get into contact with the skin. On the other hand, the porous column is a tapered column, whose end face in contact with an organism is relatively small, allowing more accurate positioning.

2. In the bioelectrical signal sensor provided by the present disclosure, the porous column is made of a porous ceramic material. With the help of capillary force, the electrolyte release through the porous ceramic column, a small amount of electrolyte wets the skin, there is not a lot of liquid or gel spreading, there will be no short circuit with other electrode sites, and a good ionic path is provided. Compared with dry electrode that does not use any electrolyte, the bioelectrical signal sensor provided by the present disclosure has low and stable electrode-skin impedance, can accurately record EEG signals, and has high measurement accuracy, low noise and artifacts. The porous ceramic column is a rigid column and does not need additional support, thus avoiding impedance instability due to the too small ionic path caused by the support. The ionic path is relatively large, and has a relatively large area in contact with the skin, with the electrolyte supplementation also relatively rapid. In addition, when the pores are clogged by contamination, a knife can be used to remove the contamination.

3. In the bioelectrical signal sensor provided by the present disclosure, the sealing cover or the porous column is fixedly and detachably connected, which facilitates cleaning the electrolyte cavity. Besides, when the electrode is used in the hospital, especially in an emergency room, in order to prevent cross-contamination, the porous column in direct contact with the skin is detachably connected and can be made into disposable supplies.

4. The bioelectrical signal sensor provided by the present disclosure, easy to clean, can be cleaned by being just immersed in water. The bioelectrical signal sensor can absorb the electrolyte after use as long as it is immersed in the electrolyte, so as to restore to the original full state.

5. The bioelectrical signal sensor provided by the present disclosure, compared with the wet electrode, is not necessary to apply the electrically conductive adhesive to the skin surface, easy and comfortable to use, expanding the scope of application of the electrode, such as being applicable to the home EEG feedback rehabilitation treatment without the need for applying the conductive gel by professional staff. It can also be widely used in EEG related cognitive psychology, brain-computer interface, mobile medical treatment, rehabilitation training, games, wearable equipment and so on.

### BRIEF DESCRIPTION OF DRAWINGS

FIG. 1 is a schematic cross-sectional view, in which the sealing cover of the sealed end of the cavity is hermetically and detachably connected to the body of the cavity, and the electrolyte inlet end of the porous column is fixed to the mounting hole in the end face of the cavity. In FIG. 1, the detachable sealed connection of the sealing cover to the body of the cavity is a threaded connection, and the fixed connection of the electrolyte inlet end of the porous column to the mounting hole in the end face of the cavity is a one-time injection molding connection.

FIG. 2 shows a cylindrical electrolyte inlet end of the porous column, which is pressed into the mounting hole in the end face of the cavity to form the tightly fitted fixed connection.

FIG. 3 is a schematic cross-sectional view, in which the sealing cover of the sealed end of the cavity is integrally one-time formed with the body of the cavity, and the electrolyte inlet end of the porous column is detachably fixed to the mounting hole in the end face of the cavity. In FIG. 3, the electrolyte inlet end of the porous column is detachably fixed to the mounting hole in the end face of the cavity by a scarf joint.

FIG. 4 is a schematic view of the sealing cover bonded to the cavity by an adhesive, with the electrolyte inlet end of the porous column detachably fixed to the mounting hole in the end face of the cavity by threaded connection.

FIG. 5 is a schematic view of the structure of a bioelectrical signal sensor having an electrolyte supply hole in the sealing cover, with the electrode and the sealing cover hermetically fixed.

FIG. 6 is a schematic view of a groove provided in the periphery of the cavity for securing the bioelectrical signal sensor of the present disclosure to a support strip or an electrode cap.

FIG. 7 is a schematic view showing a structure in which the bioelectrical signal sensor is fixed by a positioning ring fixed to an elastic fabric, and an O-shaped tightening ring is provided between the positioning ring and the sealing cover.

FIG. 8 is a schematic view showing that the cavity is divided into an upper portion and a lower portion that are connected by a detachable threaded seal, the upper portion being integrally one-time formed with the sealing cover, and the lower portion of the cavity being fixed to the electrolyte inlet end of the porous column.

FIG. 9 is a schematic view showing that the electrode and the cavity are integrally one-time formed of an electrically insulating material, with the outer surface of the electrode and the inner surface of the cavity plated with an electrically conductive coating. After the electrode wire goes through the wall of the cavity and is hermetically fixed, the electrical signal input end of the electrode wire is communicated with the electrically conductive coating on the inner surface of the cavity.

FIG. 10 is a schematic view of a combination of the electrode and the cavity, in which the cavity is made of an electrically conductive material. The electrode wire is directly connected to the cavity, and the inner wall of the cavity is the electrode. When the cavity is made of a non-metallic electrically conductive material or an electrically insulating material, the electrode is an electrically conductive end of the electrode wire exposed from the inner wall of the cavity,

FIG. 11 is a schematic cross-sectional view of a bioelectrical signal sensor with a plurality of porous columns on the end face of the cavity.

FIG. 12 is a schematic view showing a plurality of porous columns uniformly distributed on the end face of the cavity with the centroid of the end face of the cavity as the center.

FIG. 13 is a schematic view showing a plurality of porous columns uniformly distributed on the end face of the cavity.

Reference numerals in the drawings denote the following components: 1. an electrode; 2. an electrolyte; 3. a cavity; 3′. a body; 3.1. a lower portion of the cavity; 3.2. an upper portion of the cavity; 4. a porous column; 5. a sealing cover; 6. an electrode wire; 7. a female thread; 8. a male thread; 9. an adhesive; 10. an electrolyte supply hole; 11. a hole lid; 12. a groove; 13. a support of a bioelectrical signal sensor; 14. a positioning ring; 15. a seal ring; 16. a tightening ring; and 17. an electrically conductive coating.

### DETAILED DESCRIPTION OF PREFERRED EMBODIMENTS

The present disclosure will be further described below with reference to drawings and various examples.

Example 1 is a basic example of the bioelectrical signal sensor of the present disclosure. As shown in the drawings, a bioelectrical signal sensor is provided, comprising an electrode 1, electrolyte 2, and a cavity 3 for containing electrolyte, as well as a porous column 4. The cavity 3 has a sealed end at one end and communicates at an opposite end with an electrolyte inlet end of the porous column 4, with an opposite end of the porous column 4 being a working end in contact with an organism. At least a portion of the electrode 1 is immersed in the electrolyte 2. The electrode 1 is an electrical conductor, and the cavity 3 is an electrical conductor or an electrical insulator.

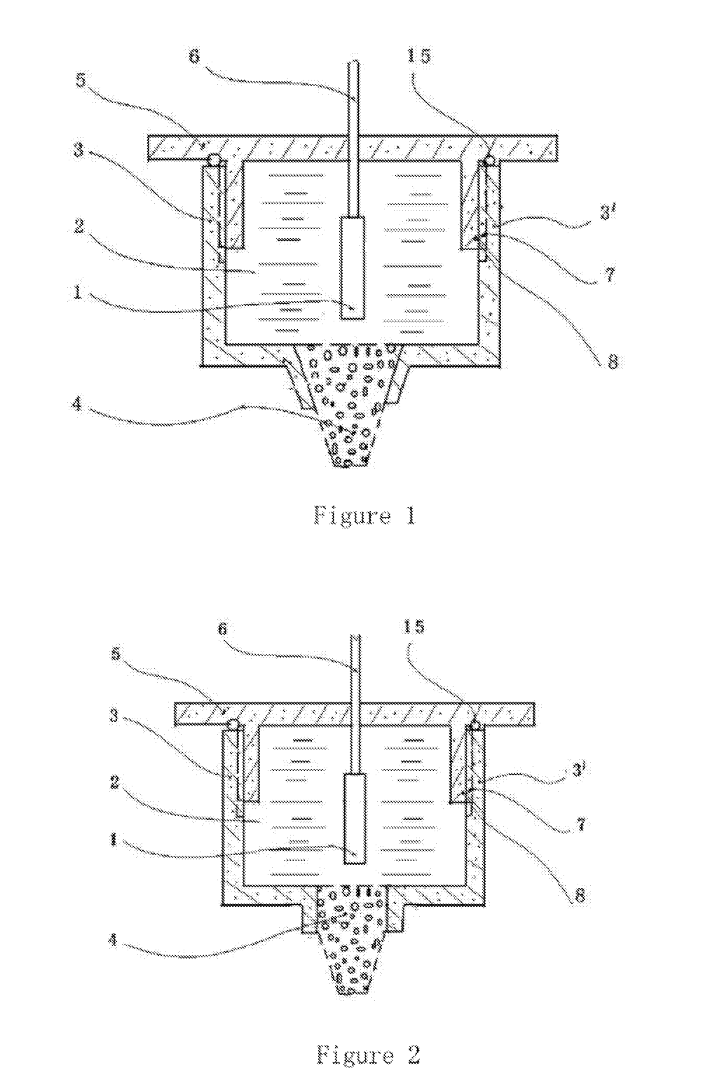

Example 2 is a derivative example or variation of Example 1. As shown in FIGS. 1-10, the bioelectrical signal sensor has one porous column. There may be more than one porous columns, as shown in FIGS. 11, 12 and 13. The porous columns are uniformly distributed on the end face of the cavity 3 with the centroid of the end face of the cavity as the center, and can also be uniformly distributed on the end face of the cavity in parallel. There is a plurality of porous columns on the end face of the cavity, and a good ionic channel is established with the electrode through the electrolyte released from the respective porous columns by capillarity, so the recorded bioelectrical signal equivalent to the sum of what are obtained from the individual porous columns to provide good measurement signal quality. In the bioelectrical signal sensor, each porous column 4 is made of a porous ceramic material or a porous ceramic composite. The porous ceramic composite is a porous ceramic material whose surface is modified with organic functional groups. Preferably, the porous ceramic is selected from alumina ceramics, silicon oxide ceramics and silicon carbide ceramics.

The advantages of the porous ceramic material are multi-folds. Firstly, the pores of the porous ceramic column can absorb the electrolyte to form an ionic conductor path connecting the scalp skin, to keep the electrode-skin impedance low and stable. This results in low bioelectrical measurement noise and stable measurement signal. Secondly, the porous column made of ceramics is a rigid solid, and easy to penetrate through the thick hair to contact the skin in the EEG measurement, such that a small amount of electrolyte liquid can provide a good ionic conductive path. In the prior art, water-absorbing fabrics or water-absorbing fibers, hydrogels and the like, which are not rigid materials, while can absorb the electrolyte, but cannot penetrate through the hair like the rigid column and thus are impossible to form a stable ionic conducive path. Thirdly, the porous ceramic column is a rigid column and does not need additional support, thus avoiding impedance instability due to the too-small ionic path caused by the support. The ionic path is relatively large, and has a relatively large contact area with the skin, with the electrolyte supplementation also relatively rapid. Fourthly, when the pores are clogged by contamination, a knife can be used to remove the contamination. Fifthly, the porous ceramic materials can adjust the rate/speed of release of the electrolyte liquid with different sizes and/or numbers of the pores.

Example 3 is a further derivative example or variation of Example 1. As shown in FIG. 1, in the bioelectrical signal sensor, the sealed end of the cavity 3 is a sealing cover 5, which is hermetically and detachably fixed to a body 3′ of the cavity 3. As shown in FIGS. 1, 2, 5, 6, 7 and 11, the sealing cover 5 is connected to the body 3′ of the cavity 3 by a threaded connection, and the electrolyte inlet end of each porous column 4 is fixed to a mounting hole in the end face of the cavity 3. In this example, the electrolyte inlet end of each porous column 4 and the mounting hole in the end face of the cavity 3 are connected by one-time injection molding.

The connection of the electrolyte inlet end of each porous column 4 to the mounting hole in the end face of the cavity 3 further has an equivalent example. As shown in FIG. 2, the porous column 4 has an electrolyte inlet end in a cylindrical shape, and may also be formed to have a cylindrical shape integrally. The electrolyte inlet end of the porous column 4 is pressed into the mounting hole in the end face of the cavity 3 to form a tightly fitted fixed connection. The porous column(s) 4 may also be bonded to the hole wall of the mounting hole in the end face of the cavity 3, or through the threaded connection as shown in FIG. 4.

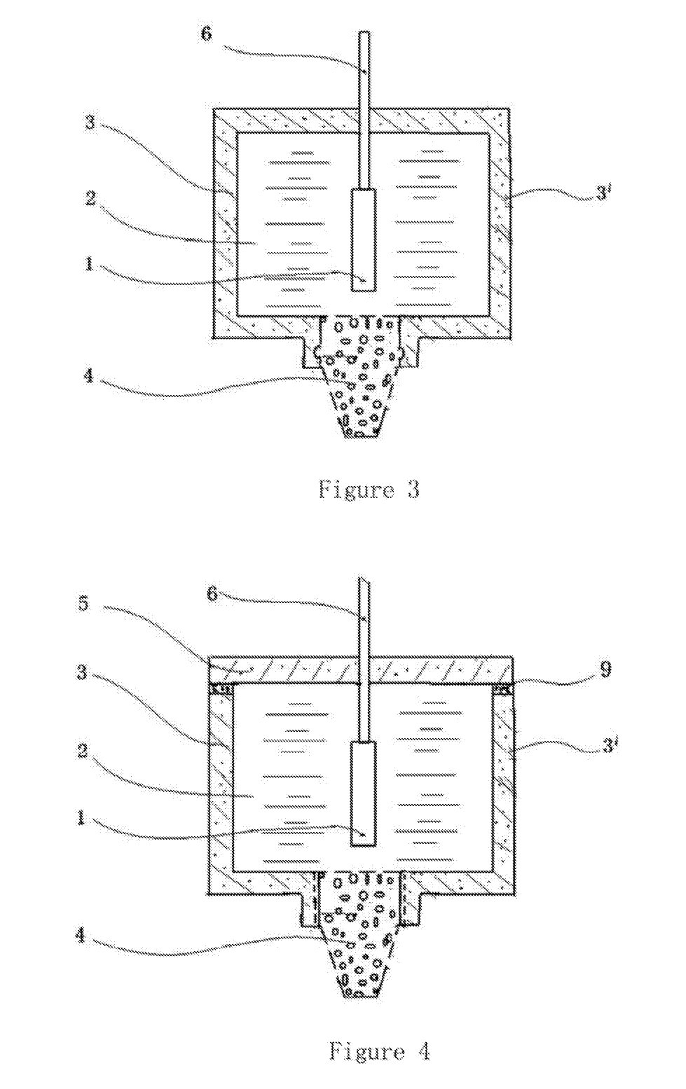

Example 4 is a further derivative example or variation of Example 1. As shown in FIG. 3, in the bioelectrical signal sensor, the sealing cover 5 of the sealed end of the cavity 3 is integrally one-time formed with the body 3′ of the cavity 3, and can also be bonded as shown in FIG. 4, and can also be welded. The electrolyte inlet end of the porous column(s) 4 is detachably fixed to the mounting hole in the end face of the cavity 3, with a scarf joint used in this example as shown in FIG. 3, or through the threaded connection as shown in FIG. 4.

In the above-described Example 3 and equivalent solution(s), when the sealed end of the cavity 3 is of a detachable fixed structure, the fixed connection of the cavity 3 to the electrolyte inlet end of the porous column(s) 4 is not limited to whether it can be detached. The sealed end is a detachable fixed structure for facilitating cleaning the electrolyte-containing cavity 3, which requires a seal ring 15 to ensure the sealed state to ensure that the electrolyte is released only from the porous column(s) 4 without letting air enter the sealed end of the cavity 3 to affect the release rate of electrolyte.

In equivalent solution(s) of above-described Example 4, when the sealed end of the cavity 3 is of a non-detachable fixed structure, the electrolyte inlet end of the porous column(s) 4 is of a detachable fixed connection, which facilitates cleaning the electrolyte-containing cavity 3. This structure has no seal ring 15, thus absolutely ensuring that the electrolyte is released only from the porous column 4 without letting air enter the sealed end of the cavity 3 to affect the release rate of electrolyte.

In some examples, the sealing cover or each porous column is fixedly and detachably connected, which has an advantage of being easy to clean. In addition, for being used in the hospital, especially an emergency room, in order to prevent cross-contamination, the porous column(s) in direct contact with the skin is/are detachably connected and can be made into disposable supplies.

Example 5 is a further derivative example or variation of Example 1. As shown in FIG. 8, in the bioelectrical signal sensor, the cavity 3 is divided into an upper portion and a lower portion that are connected by a detachable threaded seal. The upper portion 3.2 of the cavity is integrally one-time formed with the sealing cover 5. The lower portion 3.1 of the cavity is fixed to the electrolyte inlet end of the porous column. A seal ring can be used if necessary. The detachable sealing connection between the upper and lower portions of the cavity 3 is not limited to the threaded connection. In an alternative embodiment, the upper portion 3.2 of the cavity is bonded or welded to the sealing cover 5. The detachable sealing connection between the upper and lower portions has an advantage of being easy to clean. In addition, for being used in the hospital, especially an emergency room, in order to prevent cross-contamination the lower portion of the cavity in direct contact with the skin is detachably connected and can be made into disposable supplies.

Example 6 is a further derivative example or variation of Example 1. As shown in FIG. 9, in the bioelectrical signal sensor, the electrode 1 and the cavity 3 are integrally one-time formed of an electrically insulating material. The inner surface of the cavity 3 and the surface of the electrode 1 are plated with an electrically conductive coating 17. The electrode wire 6 traverses through the wall of the cavity 3 and then is hermetically fixed, the electrical signal input end of the electrode wire 6 is communicated with the electrically conductive coating on the inner surface of the cavity 3, with the electrode 1 being an electrical conductor connected to the electrically conductive inner wall of the cavity 3 and a boss in the cavity.

Example 7 is a further derivative example or variation of Example 1. As for the electrode, as shown in FIGS. 1-8 and 11, the electrode 1 is an independent electrical conductor connected with an electrode wire 6.

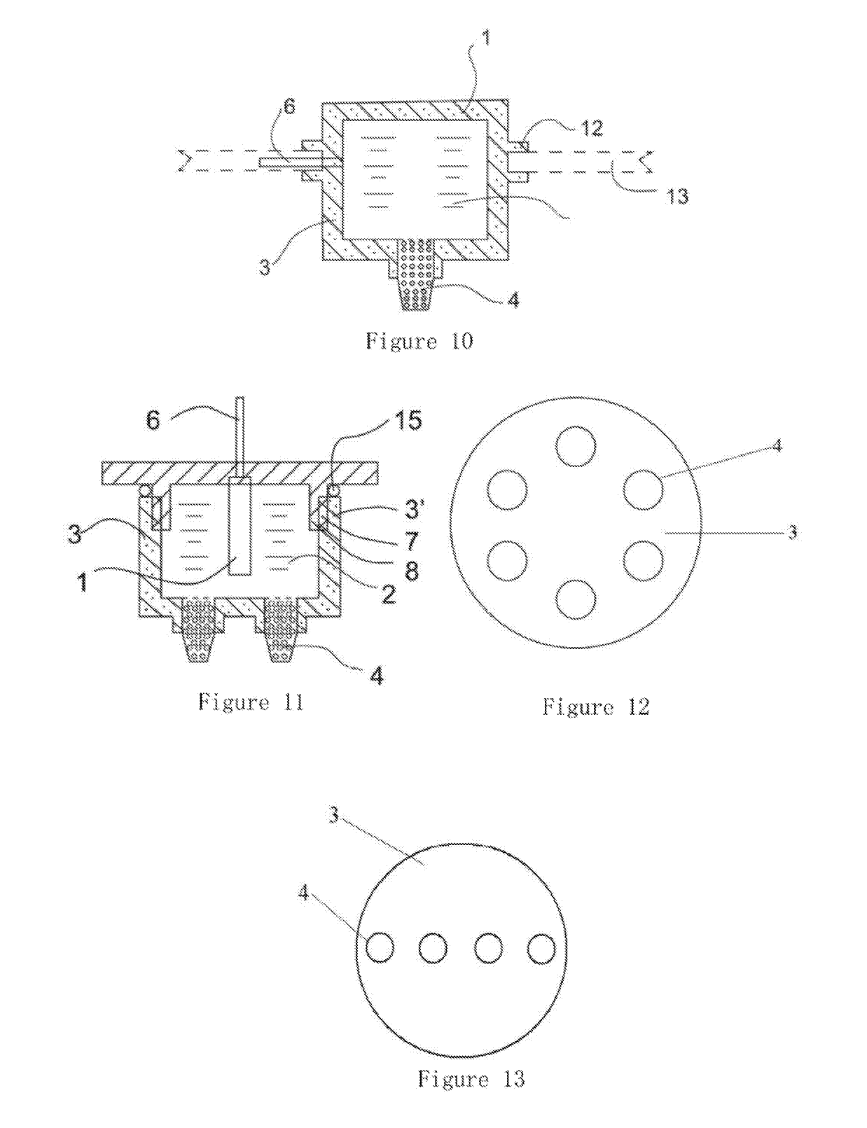

Further, as shown in FIG. 10, the electrode 1 and the cavity 3 are combined. The cavity 3 is made of an electrically conductive material, and the electrode wire 6 is directly connected to the cavity 3. The inner wall of the cavity 3 is the electrode 1.

In some embodiments, when the cavity 3 as shown in FIG. 10 is made of a non-metallic electrically conductive material or an electrically insulating material, the electrode 1 is an electrically conductive end of the electrode wire 6 exposed from the inner wall of the cavity 3.

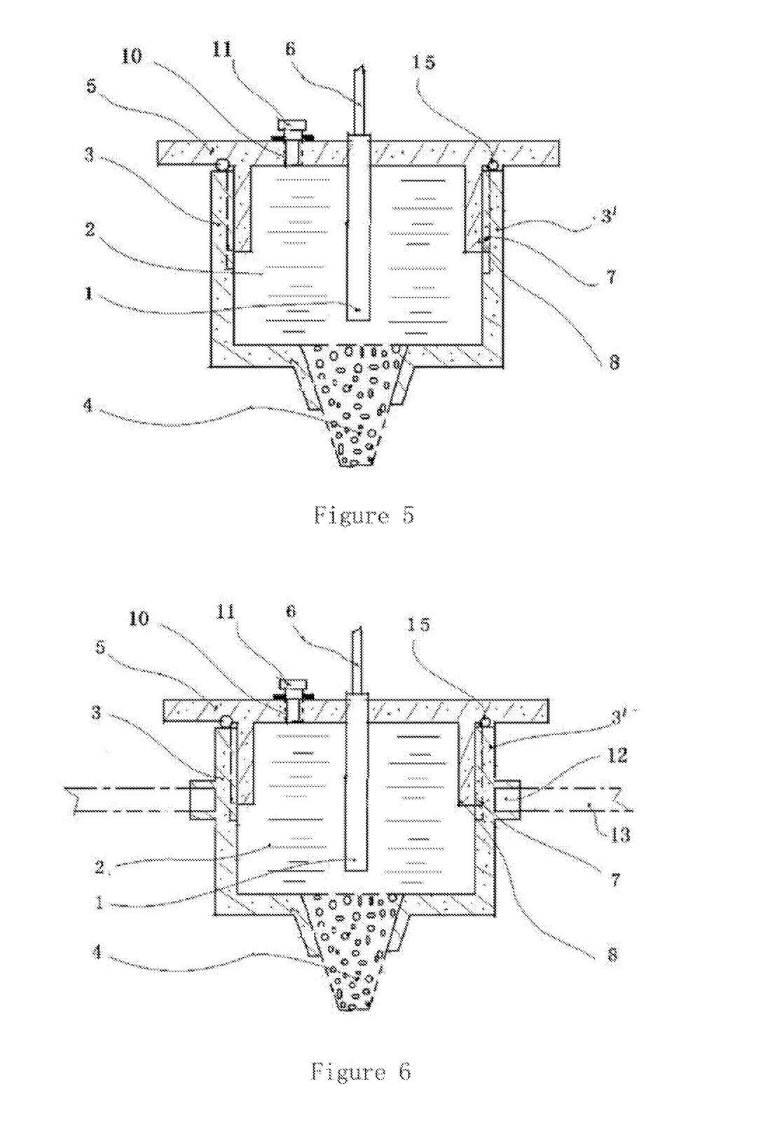

A part of the electrode 1 (e.g., as shown in FIGS. 5, 6, 8 and 11) is hermetically fixed to the cavity 3, or as the electrode wire 6 (as shown in FIGS. 1, 2, 3, 4, 7, 9 and 10) is hermetically fixed to the cavity 3, and other part or all the other part of the electrode 1 is in contact with the electrolyte 2.

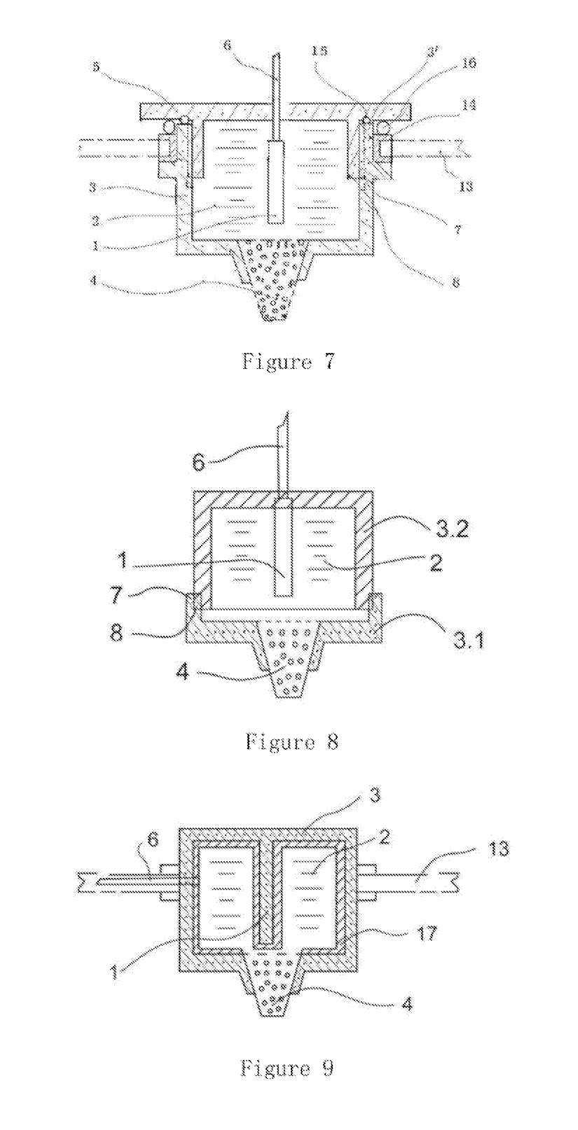

As shown in FIGS. 9 and 10, the electrode wire 6 in Examples 6 and 7 can be directly connected to the groove 12 of the cavity 3, so as to facilitate being arranged on a support 13 of the bioelectrical signal sensor. When there is no groove 12 on the exterior of the cavity 3, the electrode wire 6 may be in communication with an external power-line on the positioning ring 14 through an electrically conductive element such as a terminal on the cavity 3 as shown in FIG. 7.

Example 8 is a further derivative example or variation of Example 1. As shown in FIGS. 1-11, in the bioelectrical signal sensor, each porous column 4 is a tapered column, whose working end in contact with an organism is smaller than the electrolyte inlet end. The tapered column is different from the cylindrical column in that, a tapered column can go through the hair like a comb, thus overcoming the impact of hair, to ensure good contact with the skin such that a stable ionic path can be established. Besides, the end face of the tapered column in contact with an organism is relatively small, thereby allowing more accurate positioning.

Example 9 is a further derivative example or variation of Example 1. As shown in FIGS. 5 and 6, in the bioelectrical signal sensor, the sealing cover 5 is provided with an electrolyte supply hole 10 and a hole lid 11, which are conducive to replenishment for some bioelectrical signal sensors in work due to too fast release, and can also be used for replenishment of electrolytes after operation of the bioelectrical signal sensor. Besides, generally, as long as the bioelectrical signal sensor is immersed in the electrolyte, the porous column 4 can absorb the electrolyte after a certain period of time to restore to an original full state. In addition, the electrolyte supply hole 10 and the hole lid 11 are convenient for observing the amount of the electrolyte for replenishment of the electrolyte through the electrolyte supply hole in time.

Example 10 is a further derivative example or variation of Example 1. As shown in FIGS. 5 and 6, in the bioelectrical signal sensor, the electrode 1 is fixed to the sealed end of the cavity 3 by injection molding. Alternatively, as shown in FIGS. 1, 2, 3, 4 and 7, the electrode wire 6 of the electrode 1 is fixed to the sealed end of the cavity 3.

Example 11 is a further derivative example or variation of Example 1. Each porous column 4 of the bioelectrical signal sensor is provided on the working end in contact with an organism with a soft cushion, whose area is approximately equal to the area of the working end of each porous column 4 in contact with an organism, with the soft cushion material selected from sponge or cotton fabrics. The cushion has an effect that the porous columns 4 can be brought into contact with the skin in a soft and comfortable manner, particularly suitable for newborns, infants and children.

Example 12 is a further derivative example or variation of Example 1. In the bioelectrical signal sensor, the electrical conductor is a conductor made of an electrically conductive material, or an electrically insulating material plated on the surface with an electrically conductive material, or a composite conductor of electrically conductive material(s) and electrically insulating material(s). A composite conductor of electrically conductive material(s) and an electrically insulating material(s) can be obtained as follows: The electrically conductive material is mixed with the electrically insulating material, or the electrically conductive material is filled or dispersed in the electrically insulating material to form a conductor, e.g., a metallic material is evenly filled and dispersed in the electrically insulating material of silicone rubber to form an electrically conductive silicone rubber conductor. The electrically conductive material is selected from gold, silver, silver/silver chloride, electrically conductive silicone rubber, conductive polymer, conductive carbon material, and composite material thereof. The electrically insulating material is selected from plastic, rubber, and composite material thereof. The electrolyte 2 is conductive liquids, conductive gels, hydrogels, the conductive liquid absorbed into the sponge, or a combination thereof. Preferably, the electrolyte is conductive liquid containing sodium chloride and/or potassium chloride. The electrolyte may further include surfactant(s), so as to enhance the ability of the electrolyte wetting the skin. When antibacterial ingredients are contained, a disinfectant antibacterial effect can be achieved.

In the above examples, in order to support the bioelectrical signal sensor of the present disclosure in use, the cavity 3 is fixed to the support 13 of the bioelectrical signal sensor, which is an electrode cap, an electrode vest, or an electrode wristband. As shown in FIG. 6, the cavity 3 is provided on the outer wall with a groove 12, such that the peripheral support 13 of the bioelectrical signal sensor is fitted into the groove 12 for positioning. Alternatively, as shown in FIG. 7, a positioning ring 14 is fitted around the cavity 3, and provided on the periphery with a groove that is matched with the mounting hole in the support 13 of the bioelectrical signal sensor. An O-shaped tightening ring 16 is arranged between the positioning ring 14 and the sealing cover.

The scope of protection of the present disclosure is not limited to the above illustrative examples.

1. A bioelectrical signal sensor, comprising:
an electrode;
an electrolyte;
a cavity capable of containing electrolyte; and
one or more porous columns,
wherein the cavity has a sealed end at one end and an opposite end communicating with an electrolyte inlet end of each of the one or more porous columns, with an opposite end of each of the one or more porous columns being a working end configured to be in contact with an organism,
wherein at least a portion of the electrode is located in the electrolyte,
wherein the electrode is an electrical conductor.

2. The bioelectrical signal sensor of claim 1, wherein the one or more porous columns are made of a porous ceramic material or a porous ceramic composite.

3. The bioelectrical signal sensor of claim 1, wherein the electrical conductor is a conductor made of an electrically conductive material, an electrically insulating material plated with an electrically conductive material, or a composite conductor of one or more electrically conductive materials and one or more electrically insulating materials.

4. The bioelectrical signal sensor of claim 1, wherein the electrode and the cavity are integrally one-time formed of an electrically conductive material, or are integrally one-time formed of an electrically insulating material plated with an electrically conductive coating.

5. The bioelectrical signal sensor of claim 1, wherein the sealed end of the cavity is a sealing cover, which is hermetically and detachably fixed to a body of the cavity, and wherein the electrolyte inlet end of each of the one or more porous columns is fixed to a mounting hole in an end face of the cavity.

6. The bioelectrical signal sensor of claim 1, wherein the sealed end of the cavity is a sealing cover, which is integrally bonded to, welded to, or one-time formed with a body of the cavity, and wherein the electrolyte inlet end of each of the one or more porous columns is detachably fixed to a mounting hole in an end face of the cavity by a scarf joint or a threaded connection.

7. The bioelectrical signal sensor of claim 1, wherein the cavity is divided into an upper portion and a lower portion that are connected by a detachable seal, wherein the upper portion of the cavity is integrally bonded to, welded to, or one-time formed with a sealing cover, and wherein the lower portion of the cavity is fixed to the electrolyte inlet end of each of the one or more porous columns.

8. The bioelectrical signal sensor of claim 1, wherein each of the one or more porous columns is a tapered column, with the working end thereof in contact with the organism being smaller than the electrolyte inlet end thereof.

9. The bioelectrical signal sensor of claim 1, wherein a sealing cover is provided with an electrolyte supply hole and a hole lid.

10. The bioelectrical signal sensor of claim 1, wherein the electrode is an independent electrical conductor connected with an electrode wire, an electrically conductive end of the electrode wire exposed from an inner wall of the cavity, or an electrically conductive inner wall of the cavity to which an electrical wire is connected, and wherein a part of the electrode or the electrode wire is hermetically fixed on the cavity, with another part of the electrode being in contact with the electrolyte.

11. The bioelectrical signal sensor of claim 1, wherein the electrode is fixed to the sealed end of the cavity by injection molding, or is bonded to the sealed end of the cavity.

12. The bioelectrical signal sensor of claim 3, wherein the electrically conductive material comprises gold, silver, silver/silver chloride, electrically conductive silicone rubber, conductive polymer, an electrically conductive carbon material, or a composite material thereof.

13. The bioelectrical signal sensor of claim 3, wherein the electrically insulating material comprises plastic, rubber, or a composite material thereof.

14. The bioelectrical signal sensor of claim 1, wherein the electrolyte comprises a conductive liquid, a conductive gel, or a combination thereof.

15. The bioelectrical signal sensor of claim 1, wherein the cavity is an electrical conductor.

16. The bioelectrical signal sensor of claim 1, wherein the cavity is an electrical insulator.
